(19) United States
(12) Patent Application Publication
(10) Pub. No.: US 2007/0232947 A1
(43) Pub. Date: Oct. 4, 2007

(54) Method of extracting low-frequency or high-frequency component from a signal with slope tracing waves

(76) Inventors: Jung-Kuk Kim, Sung-nam (KR); Ki-Young Lee, Sung-nam (KR); Jae-Hyun Park, Sung-nam (KR); Min-Kyu Kim, Suwon (KR)

Correspondence Address: MARTINE PENILLA & GENCARELLA, LLP, 710 LAKEWAY DRIVE, SUITE 200, SUNNYVALE, CA, 94085, US

(21) Appl. No.: 11/701,097

(22) Filed: Jan. 31, 2007

(30) Foreign Application Priority Data
Feb. 2, 2006 (KR) 2006-0009944

(51) Int. Cl. A61B 5/04

(52) U.S. Cl. 600/509

(57) ABSTRACT

This invention is about extracting two signals from the signal in which more than two frequency components are mixed. In detail, this is about extracting low or high frequency component by distinguishing signal distortion using slope tracing wave. This invention applies two slope tracing wave to an arbitrary signal to track signal distortion and extract and remove low frequency component such as the variation of electrocardiogram signal baseline or easily extract or remove 60 Hz interference wave easily introduced to electrocardiogram. In addition, this allows easy detection of a specific waveform such as P wave and T wave from the electrocardiogram diagram by using the difference between the arbitrary signal and two slope tracing wave. The methods of this invention include distinguishing signal distortion, and detecting, extracting, removing the variation of unfavorable baseline using the shape characteristics of the distinguished section, and also detecting, extracting, removing external interference wave such as 60 Hz noise. In addition, the methods of this invention include distinguishing signal distortion and easily detecting P wave and T wave when the baseline is changed using the shape characteristics of the distinguished section.

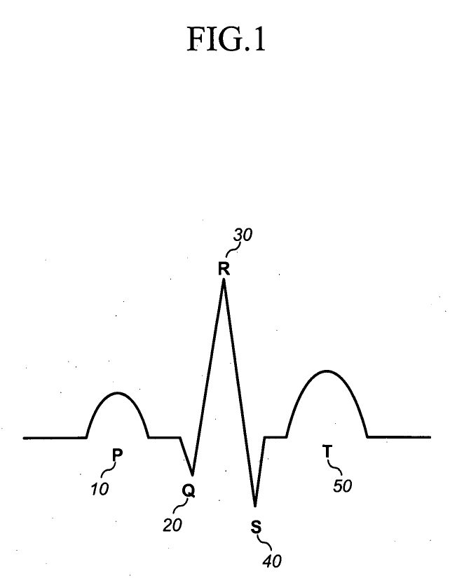

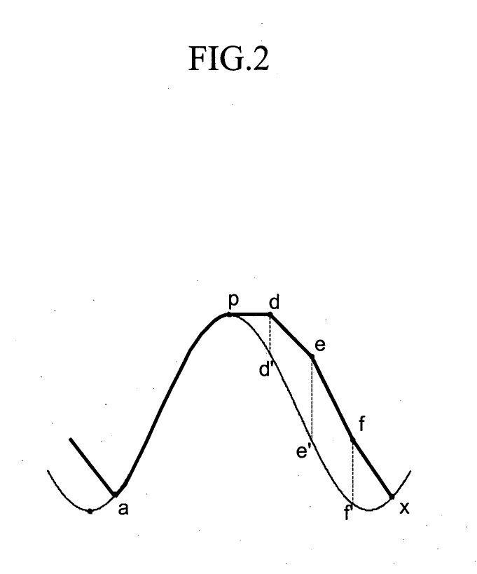

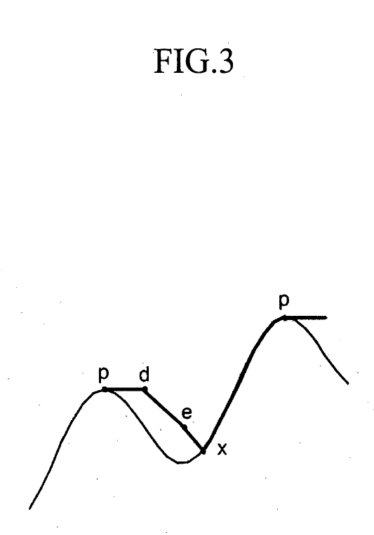

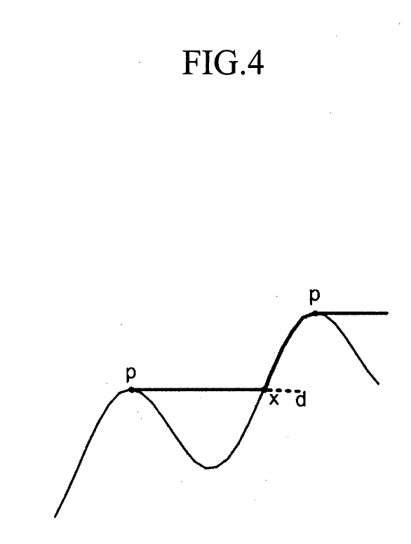

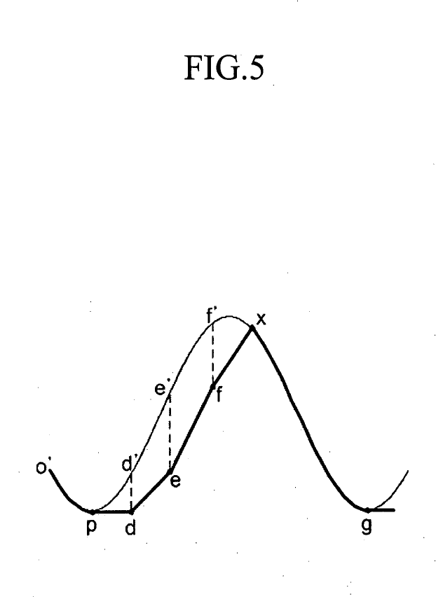

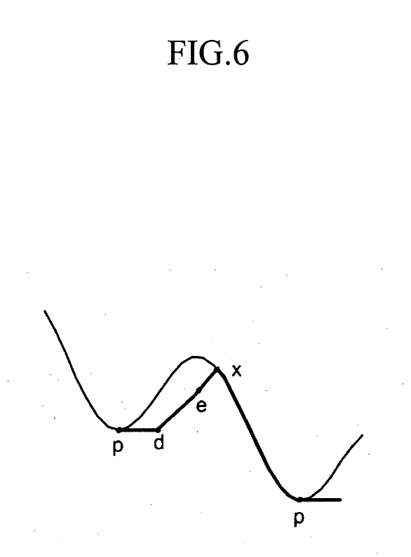

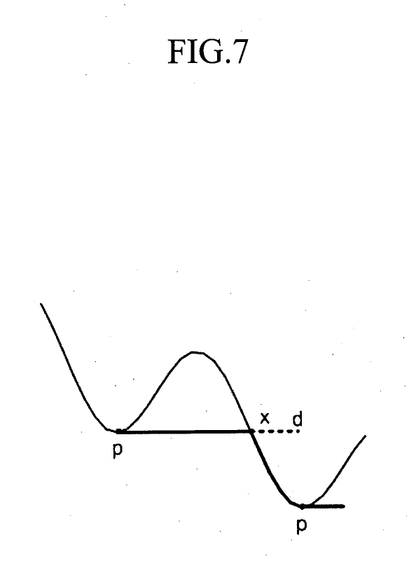

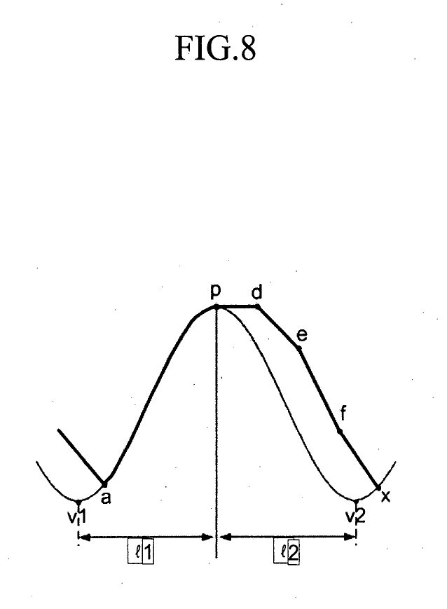

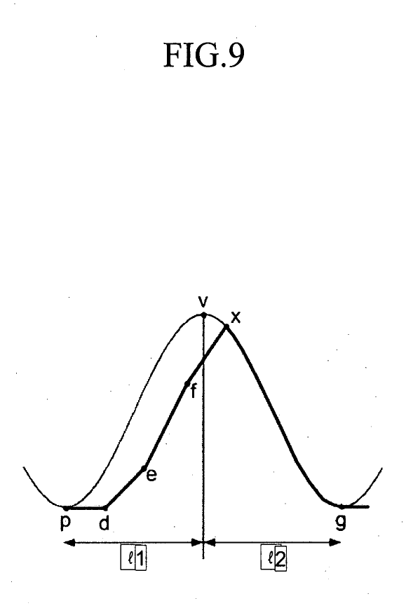

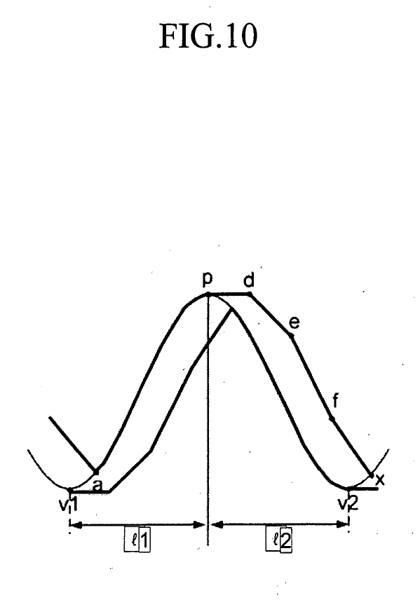

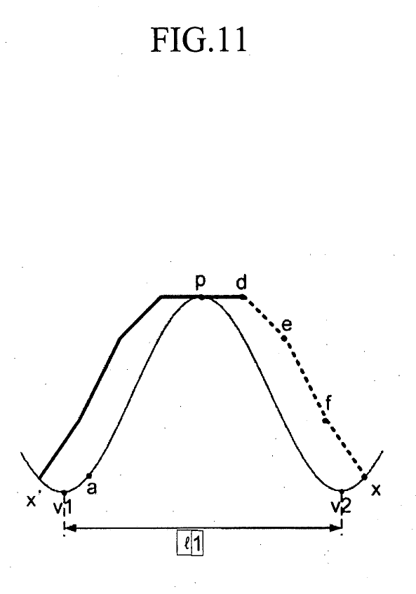

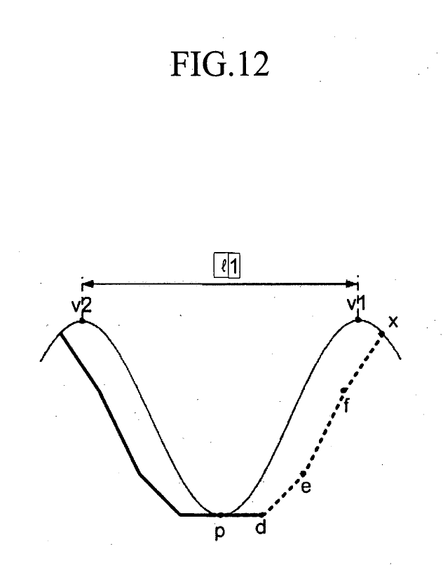

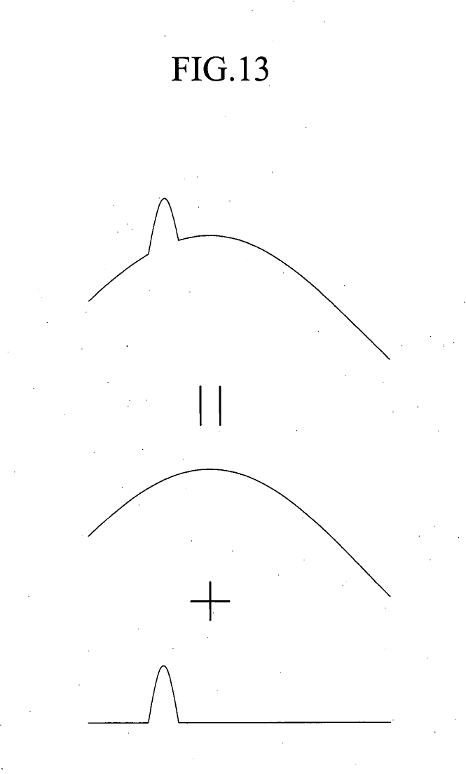

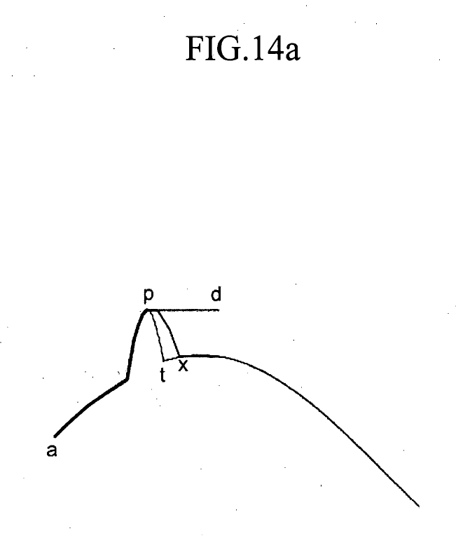

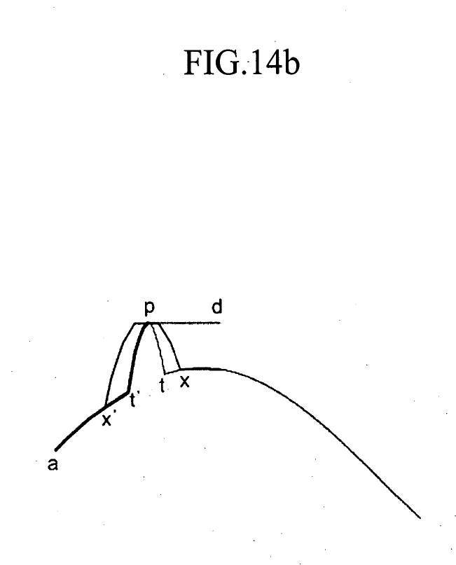

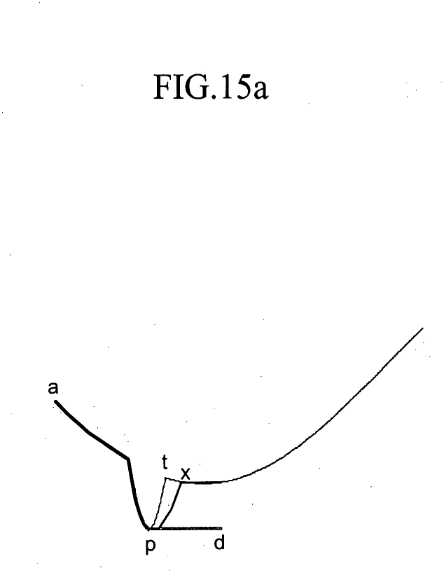

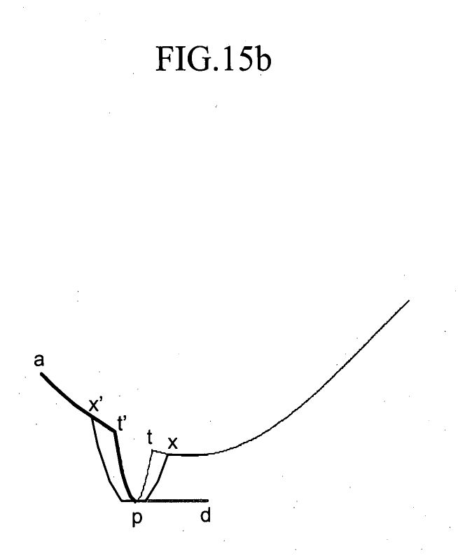

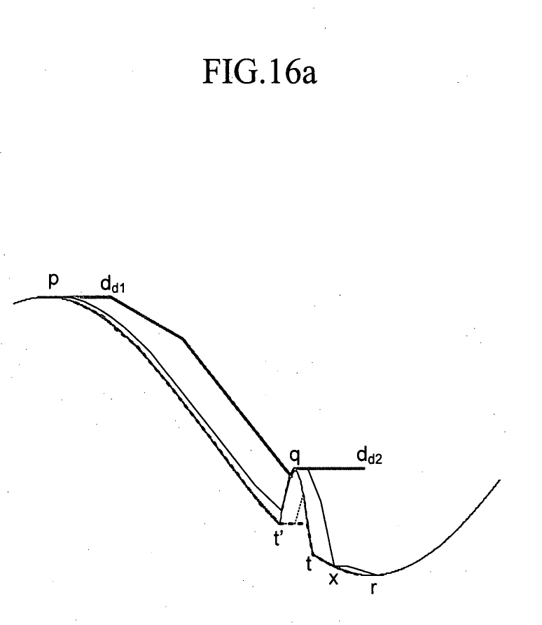

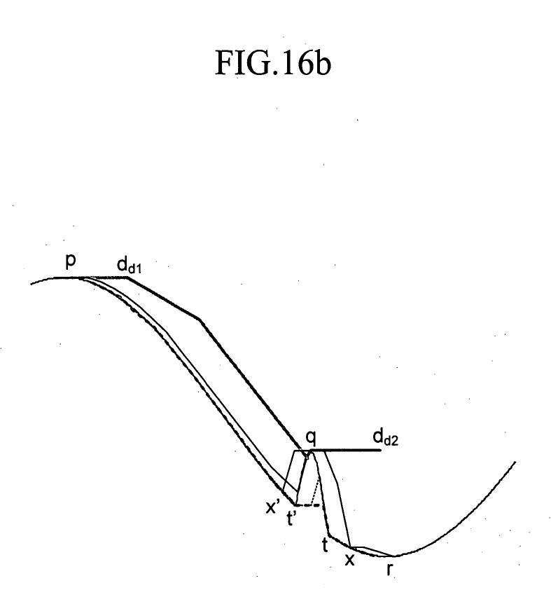

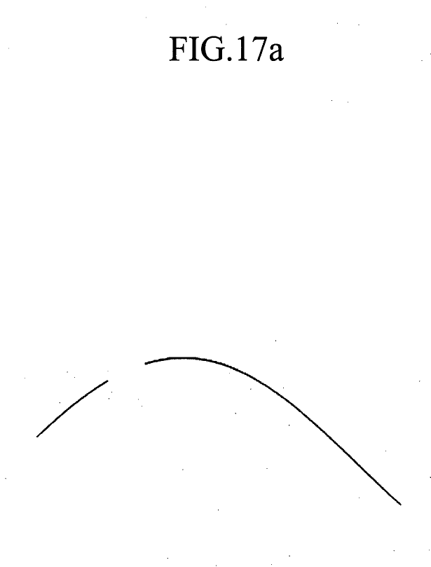

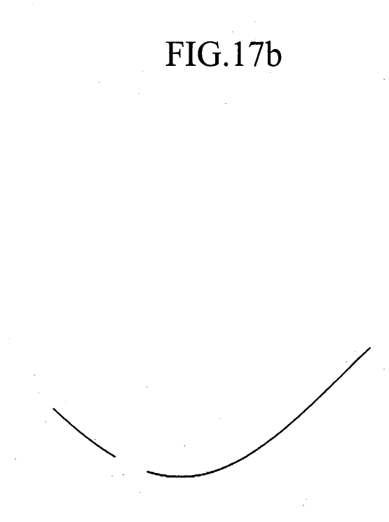

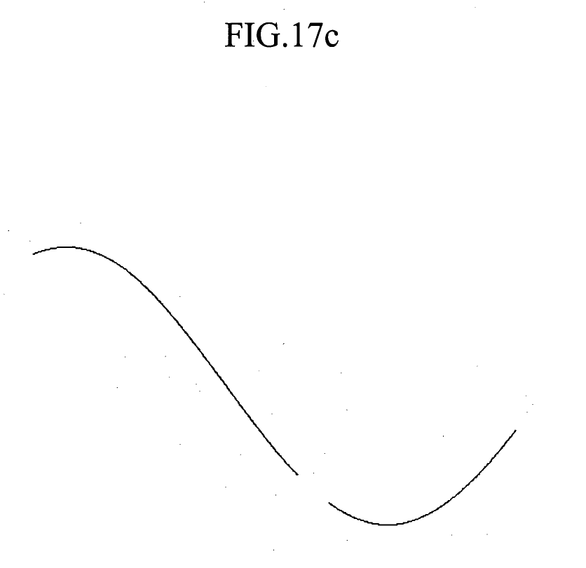

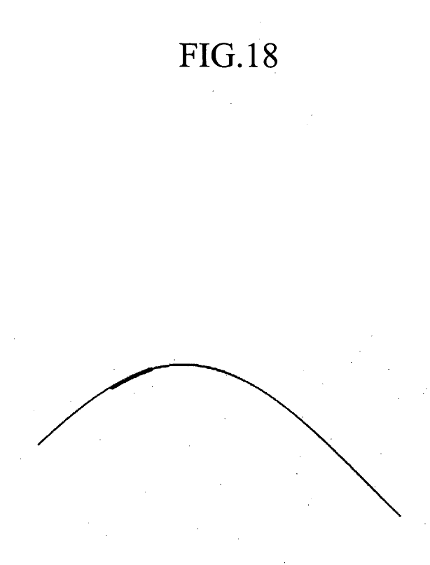

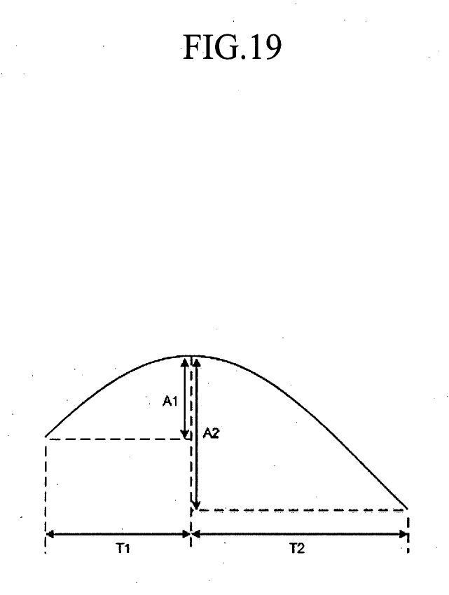

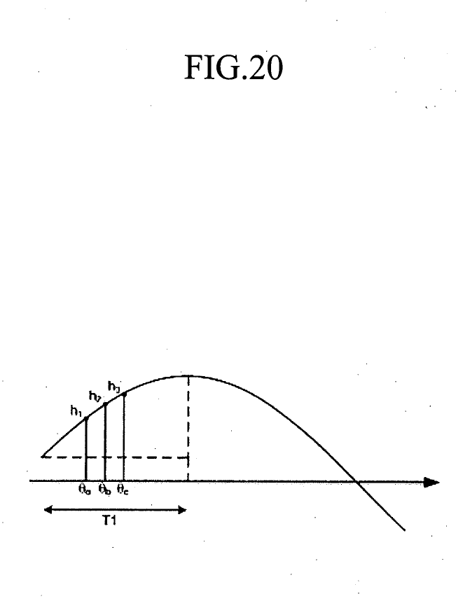

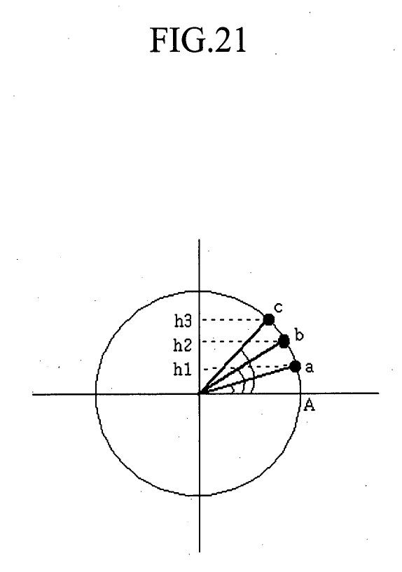

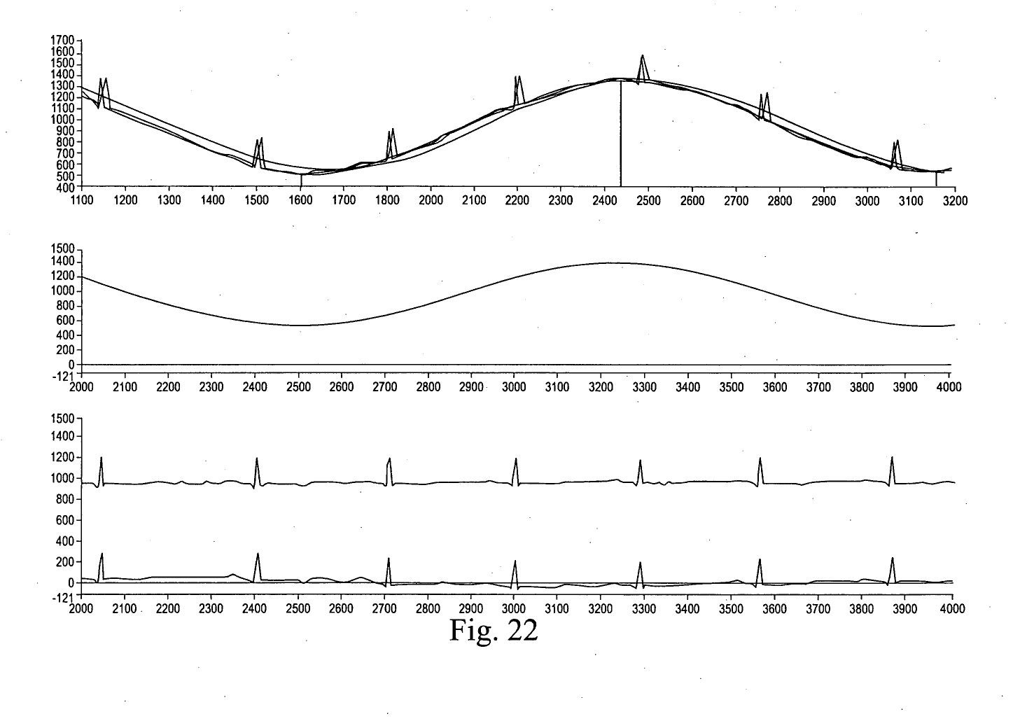

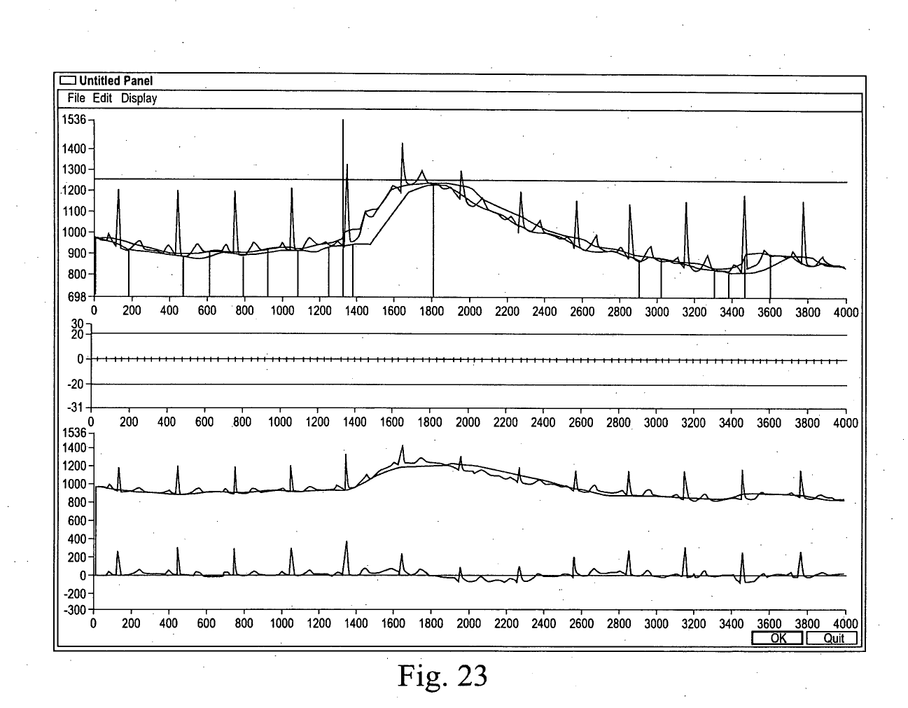

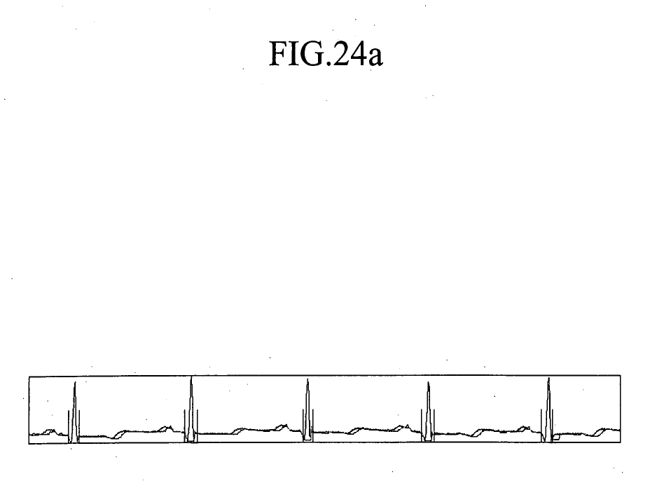

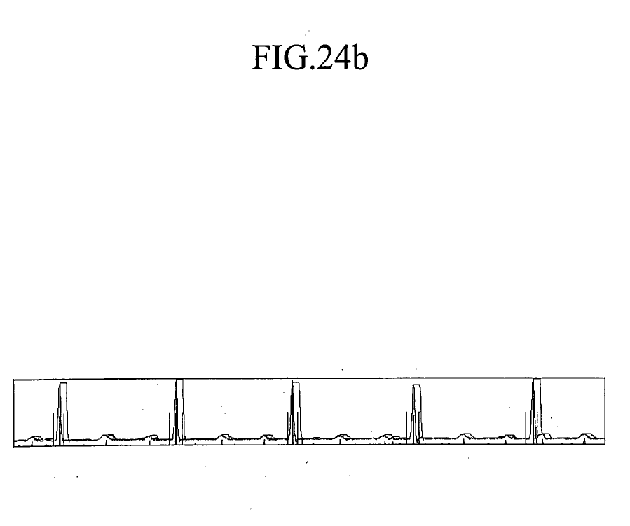

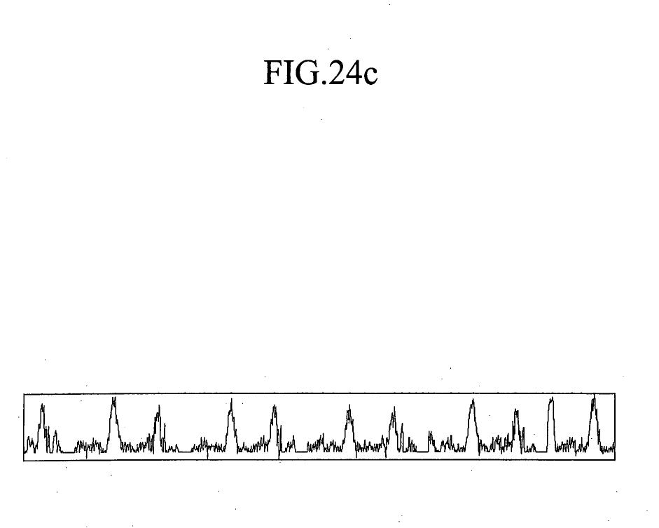

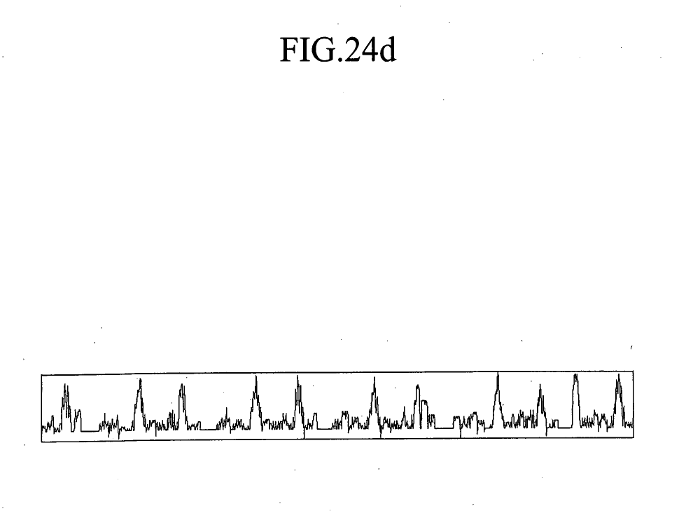

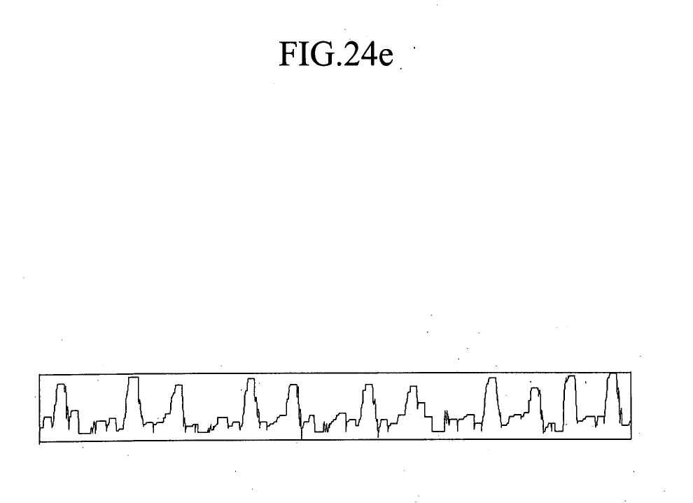

### CLAIM OF PRIORITY

This application claims priority under 35 U.S.C. §119 from Korean Patent Application No. 2006-0009944, filed Feb. 2, 2006.

### BACKGROUND

As a traditional method used to detect P wave and T wave of electrocardiogram, a method detecting and removing QRS wave from electrocardiogram waveform and setting up search section for the signal out of the band-pass filter had been proposed by Hengebeld and Bemmel in 1976 (pp.125˜132 Vol.9 Journal of Computers and Biomedical Research.)

In addition, Gritzali, et al. proposed a method applying an algorithm to the differential value of multiple channel signals which are coupling length converted (pp.83˜91 Vol.22 Journal of Computers and Biomedical Research.)

However, the above-mentioned traditional methods require complex calculation and the calculation amount is excessive, and its method and implementation are tricky because it requires too many filters when electrocardiogram baseline is fluctuated or noise such as 60 Hz interference wave is introduced.

### BRIEF DESCRIPTION OF THE DRAWINGS

Drawing 1 shows a usual electrocardiogram waveform.

Drawing 2 shows a descending slope tracing wave tracing an arbitrary sinusoidal signal according to this invention.

Drawing 3 shows the operation change of descending slope tracing wave depending on sample number k when the p-d section of slope tracing wave is short.

Drawing 4 shows the operation change of descending slope tracing wave depending on sample number k when the p-d section of slope tracing wave is long.

Drawing 5 shows a ascending slope tracing wave tracing an arbitrary sinusoidal signal according to this invention.

Drawing 6 shows the operation change of ascending slope tracing wave depending on sample number k when the p-d section of slope tracing wave is short.

Drawing 7 shows the operation change of ascending slope tracing wave depending on sample number k when the p-d section of slope tracing wave is long.

Drawing 8 shows an example determining signal section using descending slope tracing wave.

Drawing 9 shows an example determining signal section using ascending slope tracing wave.

Drawing 10 shows an example determining signal distortion section using both descending slope tracing wave and ascending slope tracing wave.

Drawing 11 shows an example determining distortion section by generating reverse descending slope tracing wave at its max. p point before generating next signal sample when descending slope tracing wave arrives at point d.

Drawing 12 shows an example determining distortion section by generating reverse ascending slope tracing wave at its max. p point before generating next signal sample when ascending slope tracing wave arrives at point d.

Drawing 13 shows mixed two signals in which high frequency signal is overlapped on low frequency sinusoidal signal.

Drawing 14a shows the first step of the algorithm extracting a signal using two descending slope tracing waves.

Drawing 14b shows the second step of the algorithm extracting a signal using two descending slope tracing waves.

Drawing 15a shows the first step of the algorithm extracting a signal using two ascending slope tracing waves.

Drawing 15b shows the second step of the algorithm extracting a signal using two ascending slope tracing waves.

Drawing 16a shows the first step of the algorithm extracting a signal using two descending slope tracing waves and two ascending slope tracing waves.

Drawing 16b shows the second step of the algorithm extracting a signal using two descending slope tracing waves and two ascending slope tracing waves.

Drawing 17a, 17b, or Drawing 17c shows the waveform which is obtained by removing high frequency components from the original signal.

Drawing 18 shows low frequency waveform recovered from the segmented waveform in Drawing 17a. through interpolation.

Drawing 19 shows the approximation of extracted waveform to two different sinusoidal wave.

Drawing 20 shows an example of the approximation to sinusoidal wave using three extracted points.

Drawing 21 shows a successful example of the approximation to sinusoidal wave using three extracted points.

Drawing 22 and Drawing 23 show an example removing low frequency component from electrocardiogram diagram in accordance with the method of this invention.

Drawing 24a shows the original electrocardiogram signal s(n) and a(n).

Drawing 24b shows D_a_s(n) and descending slope tracing wave d(n) in the electrocardiogram graph.

Drawing 24c shows D_a_s(n) of this invention and QRS wave determined by the above-mentioned method to emphasize P wave and T wave from the diagram.

Drawing 24d shows a diagram where QRS wave is removed by the method above-mentioned in the signal difference given in the equation 13 of this invention.

Drawing 24e shows a diagram where the signals obtained in this invention are shown.

### SUMMARY

This invention uses two descending slope tracing waves which have two different value k1 and k2 (k1<k2), i.e. the 1^{st} descending slope tracing wave d1(n) and the 2^{nd} descending slope tracing wave d2(n) to extract low frequency component and high frequency component from the s(n) which is sampled at a fixed interval for the original signal which has more than two frequency components. The method is composed of several steps as below:

(a) Check if the above-mentioned 1st descending slope tracing wave d1(n) meets signal s(n) in the section of k2 in which the above-mentioned 2^{nd} descending slope tracing wave d2(n) maintains function value when the above-mentioned signal s(n) reaches at the maximum value (p point) at the nth sample point:

(b) When there is a cross point (point x) where d1(n) and s(n) meet each other in the prior step (a), determine the max. Slope-inversion point t in the section of [n, n+k2], i.e. between point p and point x, and set this up as the right most point of the section:

(c) When there is not a cross point (point x) where d1(n) and s(n) meet each other in the prior step (a), it is assumed there is no high frequency component and the above-mentioned 1^{st} descending slope tracing wave d1(n) and the 2^{nd} descending slope tracing wave d2(n) continue slope tracing:

(d) When there is a cross point (point x) where d1(n) and s(n) meet each other in the prior step (a), reverse descending slope tracing wave is applied at the nth sample point (p point) to find x′ point where it meets with signal s(n) and determines max. Slope-inversion point t′ of a signal between x′ point and p point, and set it ups as the left most point of the section:

(e) The left and right section waveforms which are determined as the right most end and the left most end of the above-mentioned section are extracted from the original signal:

(f) The right most end and the left most end of the section are connected.

This invention uses two ascending slope tracing waves which have two different value k1 and k2 (k1<k2), i.e. the 1^{st} ascending slope tracing wave a1(n) and the 2^{nd} ascending slope tracing wave a2(n) to extract low frequency component and high frequency component from the s(n) which is sampled at a fixed interval for the original signal which has more than two frequency components, and the method is composed of several steps as below:

(a) Check if the above-mentioned 1st ascending slope tracing wave a1(n) meets signal s(n) in the section of k2 in which the above-mentioned 2^{nd} ascending slope tracing wave a2(n) maintains function value when the above-mentioned signal s(n) reaches at the maximum value (p point) at the nth sample point:

(b) When there is a cross point (point x) where a1(n) and s(n) meet each other in the prior step (a), determine the max. Slope-inversion point t in the section of [n, n+k2], i.e. between point p and point x, and set this up as the right most point of the section:

(c) When there is not a cross point (point x) where al(n) and s(n) meet each other in the prior step (a), it is assumed that there is no high frequency component and the above-mentioned 1^{st} descending slope tracing wave a1(n) and the 2^{nd} descending slope tracing wave a2(n) continue slope tracing:

(d) When there is a cross point (point x) where a1(n) and s(n) meet each other in the prior step (a), reverse descending slope tracing wave at the nth sample point (p point) is applied to find x′ point where it meets with signal s(n) and determines max. Slope-inversion point t′ of a signal between x′ point and p point, and set it ups as the left most point of the section:

(e) The left and right section waveforms which are determined as the right most end and the left most end of the above-mentioned section are extracted from the original signal:

(f) The right most end and the left most end of the section are connected. This invention uses two ascending slope tracing waves which have two different value k1 and k2 (k1<k2), i.e. the 1^{st} ascending slope tracing wave a1(n) and the 2^{nd} ascending slope tracing wave a2(n) to extract low frequency component and high frequency component from the s(n) which is sampled at a fixed interval for the original signal which has more than two frequency components, and the method is composed of several steps as below:

A method using two descending slope tracing waves which have two different value k1 and k2 (k1<k2), i.e. the 1^{st} descending slope tracing wave d1(n) and the 2^{nd} descending slope tracing wave d2(n) to extract low frequency component and high frequency component from the s(n) which is sampled at a fixed interval for the original signal which has more than two frequency components, and also using two ascending slope tracing waves which have two different value k1 and k2 (k1<k2), i.e. the 1^{st} ascending slope tracing wave a1(n) and the 2^{nd} ascending slope tracing wave a2(n) to extract low frequency component and high frequency component from the s(n) which is sampled at a fixed interval for the original signal which has more than two frequency components.

### TECHNICAL OBJECTIVES OF THE INVENTION

The first objective of this invention is to provide a method to separate each frequency component from a signal where more than two frequency components are mixed and also to remove unwanted signal.

The second objective of this invention is to provide a method to easily detect P wave and T wave with the minimum number of filters while maintaining the original signal of electrocardiogram waveform.

### DETAILED DESCRIPTION OF THE INVENTION

### DETAILED DESCRIPTION OF THE PRIOR EMBODIMENT OF THE INVENTION

This invention is about extracting each different signal from the signal in which more than two frequency components are mixed. In detail, this is about extracting low or high frequency component by distinguishing signal distortion using slope tracing wave.

For example, the low or high frequency extracting method of this invention can be used to remove baseline fluctuation of electrocardiogram or detect P wave or T wave. The method of baseline fluctuation removal of this invention is composed of several steps: distinguish signal distortion using slope tracing wave, approximate the fluctuation pattern of baseline using the shape characteristics of the distinguished section, and remove baseline fluctuation by subtracting the approximated waveform from the original waveform. In addition, because the detection method of electrocardiogram P wave and T wave distinguishes signal distortion using slope tracing wave and utilize the difference between the shape characteristics of the distinguished section and electrocardiogram waveform, it is easy to detect P wave and T wave even when there is a fluctuation of baseline which has low frequency characteristics.

As a very important waveform representing the electrical and physiological characteristics of a heart, the waveform distortion shape and position of ECC (electrocardiogram) have various clinical meanings. Especially, because the P wave and T wave of electrocardiogram have low amplitude and its waveform is obscure and time-variant, it is tricky to detect the waves using traditional methods.

Drawing 1 shows a usual electrocardiogram waveform to help understanding of this invention. According to Drawing 1, electrocardiogram waveform is composed of P wave related to atrium contraction (10), Q wave representing the lowest point of waveform (20), R wave representing the highest point of waveform (30), low point S wave (40), and T wave related to ventricular repolarization (50), and especially QRS wave means ventricle contraction. Because P wave, QRS wave, and T wave are very important waveforms representing the electrical and physiological characteristics of a heart and have various clinical meanings, medical staffs expect to detect P wave, QRS wave, and T wave.

### CONFIGURATION OF THE INVENTION

To achieve the above-mentioned objectives, this invention uses two descending slope tracing waves which have two different value k1 and k2 (k1<k2), i.e. the 1^{st} descending slope tracing wave d1(n) and the 2 descending slope tracing wave d2(n) to extract low frequency component and high frequency component from the s(n) which is sampled at a fixed interval for the original signal which has more than two frequency components, and the method is composed of several steps: (a) Check if the above-mentioned 1^{st} descending slope tracing wave d1(n) meets signal s(n) in the section of k2 in which the above-mentioned 2^{nd} descending slope tracing wave d2(n) maintains function value when the above-mentioned signal s(n) reaches at the maximum value (p point) at the nth sample point: (b) When there is a cross point (point x) where d1(n) and s(n) meet each other in the prior step (a), determine the max. Slope-inversion point t in the section of [n, n+k2], i.e. between point p and point x, and set this up as the right most point of the section: (c) When there is not a cross point (point x) where d1(n) and s(n) meet each other in the prior step (a), it is assumed that there is no high frequency component and the above-mentioned 1^{st} descending slope tracing wave d1(n) and the 2^{nd} descending slope tracing wave d2(n) continue slope tracing: (d) When there is a cross point (point x) where d1(n) and s(n) meet each other in the prior step (a), reverse descending slope tracing wave at the nth sample point (p point) is applied to find x′ point where it meets with signal s(n) and determines max. Slope-inversion point t′ of a signal between x′ point and p point, and set it ups as the left most point of the section: (e) The left and right section waveforms which are determined as the right most end and the left most end of the above-mentioned section are extracted from the original signal: (f) The right most end and the left most end of the section is connected.

Another example of this invention is as followings: this invention uses two ascending slope tracing waves which have two different value k1 and k2 (k1<k2), i.e. the 1^{st} ascending slope tracing wave a1(n) and the 2^{nd} ascending slope tracing wave a2(n) to extract low frequency component and high frequency component from the s(n) which is sampled at a fixed interval for the original signal which has more than two frequency components, and the method is composed of several steps: (a) Check if the above-mentioned 1^{st} descending slope tracing wave a1(n) meets signal s(n) in the section of k2 in which the above-mentioned 2^{nd} descending slope tracing wave a2(n) maintains function value when the above-mentioned signal s(n) reaches at the maximum value (p point) at the nth sample point: (b) When there is a cross point (point x) where a1(n) and s(n) meet each other in the prior step (a), determine the max. Slope-inversion point t in the section of [n, n+k2], i.e. between point p and point x, and set this up as the right most point of the section: (c) When there is not a cross point (point x) where a1(n) and s(n) meet each other in the prior step (a), it is assumed that there is no high frequency component and the above-mentioned 1^{st} descending slope tracing wave a1(n) and the 2^{nd} descending slope tracing wave a2(n) continue slope tracing: (d) When there is a cross point (point x) where a1(n) and s(n) meet each other in the prior step (a), reverse descending slope tracing wave at the nth sample point (p point) is applied to find x′ point where it meets with signal s(n) and determines max. Slope-inversion point t′ of a signal between x′ point and p point, and set it ups as the left most point of the section: (e) The left and right section waveforms which are determined as the right most end and the left most end of the above-mentioned section are extracted from the original signal: (f) The right most end and the left most end of the section is connected.

This invention generates a slope tracing wave which detects signal Slope-inversion point and Slope-transition point where slope change is big to remove baseline fluctuation introduced to electrocardiogram or 60 Hz noise, and detects P wave and T wave by calculating the amplitude difference among slope tracing waves. In addition, the slope tracing waves recalculated at every sampling point have two types: the descending slope tracing wave which traces a signal waveform which is lower than itself and the ascending slope tracing wave which traces a signal waveform which is higher than itself.

Hereinafter, examples detecting P wave and T wave of electrocardiogram are mainly described, but this invention can be applied to a wide range of applications as a waveform processing method. For example, the low frequency or high frequency components extraction method of this invention can be used to remove low frequency components or high frequency components from a signal where low frequency components are mixed as well as removing electrocardiogram baseline which may be caused by a patient's movement during electrocardiogram measurement.

The basic information about “slope tracing wave” mentioned in this invention is described in Korea Patent No. 399,739 registered on Nov. 13, 2001 which is also owned by the same inventor of this invention. Refer to Korea Patent No. 399,739 for more information on the terms and background information used in this invention.

### Description of Terminology

Slope-inversion point: a point of a sampled signal waveform where the waveform switches the polarity of its slope or the differential derivative either from the negative to the positive or from the positive to the negative.

Slope-transition point: a point where the slope of a signal waveform changes very rapidly. Here, the degree of the rapidness in the change of slope can be understood in a sense that the rate of slope-change at a certain point is larger than a predefined value (X%). As a preferred embodiment, X can be chosen as 50%.

Slope tracing wave: a waveform that is chasing a signal waveform and is employed for efficiently determining the slope-transition point and the slope-inversion point. Two types of slope-tracing waveform are disclosed as a preferred embodiment: one is a descending slope-tracing waveform which traces a signal waveform upward from the beneath, and the other is an ascending slope-tracing waveform which traces a signal waveform downward from the top. Preferably, both the ascending and descending slope-tracing waveforms can be simultaneously employed for partitioning the signal waveform. Depending upon a situation, either the ascending slope-tracing waveform or the descending slope-tracing waveform can be chosen.

[n,k]: This is a mathematical expression representing section n˜k.

Hereinafter, detail description on “waveform extraction method using slope tracing wave” which is the basic method of this invention will be followed in the first half part of this invention by referring to Drawing 2 or Drawing 12, and detail description on “low frequency or high frequency extraction method using slope tracing wave” which is the method proposed in this invention will be followed in the second half part of this invention by referring to Drawing 13 or Drawing 22.

The “slope tracing waves” in this invention include ascending slope tracing wave (named as ascending slope tracing wave in the Korea Patent No. 399,739) and descending slope tracing wave (named as lower slope tracing wave in the Korea Patent No. 399,739) and they are used to determine the section of a specific frequency component of a signal.

The slope tracing wave is refreshed to trace a signal whenever sample value is generated to trace a signal, and it is determined by comparing the amplitude of current sample value and the amplitude of slope tracing wave refreshed at prior sample value.

Description on the operation of descending slope tracing wave Drawing 2 shows the descending slope tracing wave tracing an arbitrary sinusoidal signal. The slope tracing wave is shown in a dark and thick line in the drawing 2. Drawing 2 shows both an arbitrary sinusoidal signal and a descending slope tracing wave which traces the sinusoidal signal.

The descending slope tracing wave is refreshed by comparing s[n] (the amplitude of current sampling value of a signal) and d[n−1] (the amplitude of descending slope tracing wave refreshed at prior sampling value) as described below: If the amplitude of current sampling value of a signal is higher than or equal to the amplitude of descending slope tracing wave refreshed at prior sampling value, i.e., s[n] >=d[n−1], the tracing wave is refreshed to the amplitude of the sampling value, i.e., d[n]=s[n]. This occurs between point a and point p shown in the drawing 2.

Meanwhile, if the amplitude of current sampling value of a signal is lower than the amplitude of descending slope tracing wave refreshed at prior sampling value, i.e., s[n]<d[n−1], the tracing wave is determined based on the position of sampling value on a signal. When signal sampling value reaches at the highest point p and the next signal sampling value is generated, it becomes s[n]<d[n−1] and the descending slope tracing wave maintains the amplitude of prior tracing wave, that is, it is determined to be d[n]=d[n−1]. This operation is shown in p-d section of drawing 2 and this is repeated as many times as predefined sampling number k. Here, k can be set up by a user in advance. After that, when signal sampling value is generated at the point d′ which is k-th sampling point after the highest value p, the algorithm calculates signal slope average Δ_{α} for the p-d′ section. The average Δ_{α} can be calculated by dividing the sum of the difference between adjacent sampling values with sampling number k, that is, Δ_{α}=[(s[n])−s[n−1])+(s[n−1])−s[n−2])+ . . . (s[n−k])−s[n−k−1])]/k. And the slope tracing wave in the next signal section d′-e′ is decreased by the absolute number of slope average |Δ_{α}| at every sampling value, that is, d[n]=d[n−1]−|Δ_{α}||. This process is repeated while k sampling values are generated and the shape of slope tracing wave is shown as d-e in the drawing 2.

The similar process is repeated until sampling value (point x in the drawing 2) is generated where the amplitude of slope tracing wave becomes equal to or lower than signal amplitude, that is, the slope average Δ_{α} is recalculated by dividing the sum of the difference between adjacent sampling values in the section d′-e′ with sampling number k at e′ where k-th sampling value is generated.

This recalculated absolute value of slope average |Δ_{α}| refreshes slope tracing wave as shown in e-f by decreasing it at every sampling value from slope tracing wave in the section e′-f′, that is, it is refreshed to d[n]=d[n−1]|Δ_{α}|. When a sampling value is generated at f′ which is the k-th sampling in the section, slope average Δ_{α} is recalculated and slope tracing wave is refreshed at every sampling by subtracting |Δ_{α}| in the next section.

Just as point x in the drawing 2, if slope tracing wave becomes equal to or lower than signal sampling value while it decreases, i.e., s[n]>=d[n−1], the slope tracing wave is refreshed to the amplitude of sampling value, i.e., d[n]=s[n].

Through the procedures described above, the algorithm sets up section a and p which is determined by slope tracing wave as one bend and sets up section p and x as another bend, and applies more sophisticated algorithm which will be described later to each section.

Meanwhile, Drawing 3 and drawing 4 shows the operation of descending slope tracing wave can be varied depending on sampling number k of the p-d section of slope tracing wave. When referring to drawing 3, the slope tracing wave meets a signal at point x by descending from the highest value p of the first bend when sampling number k is small. After that, it starts to trace the ascending signal at the right side as described above. In this case, two adjacent bends are determined as two separate bend.

However, as shown in the drawing 4, if sampling number k is big, signal amplitude becomes higher than slope tracing wave in the section p-d. In this case, slope tracing wave starts to trace signal amplitude at point x and two bends become one bend, i.e., the first bend is determined to be a part of the second bend.

### Description on the Operation of Ascending Slope Tracing Wave

Drawing 5 shows the ascending slope tracing wave tracing an arbitrary sinusoidal signal. The slope tracing wave is shown in a dark and thick line in the drawing 2. The operation of ascending slope tracing wave is the opposite of the operation of descending slope tracing wave described above. The ascending slope tracing wave is refreshed by comparing s[n] (the amplitude of current sampling value of a signal) and a[n−1] (the amplitude of ascending slope tracing wave refreshed at prior sampling value) as described below:

If the amplitude of current sampling value of a signal is equal to or lower than the amplitude of slope tracing wave refreshed at prior sampling value, i.e., s[n]=<a[n−1], the tracing wave is refreshed to the amplitude of the sampling value, i.e., a[n]=s[n]. This occurs when signal amplitude decreases as shown in the section o′-p or x-g of the drawing 5.

If the amplitude of current sampling value of a signal is higher than the amplitude of slope tracing wave refreshed at prior sampling value, i.e., s[n]>a[n−1], the tracing wave is determined based on the position of sampling value on a signal. When signal sampling value reaches at the lowest point p and the next signal sampling value is generated, it becomes s[n]>a[n−1] and the ascending slope tracing wave maintains the amplitude of prior tracing wave, that is, it is determined to be a[n]=a[n−1]. This operation is shown in the p-d section of drawing 5 and this is repeated as many times as predefined sampling number k.

After that, when signal sampling value is generated at the point d′ which is k-th sampling point after the lowest value p, the algorithm calculates signal slope average for the p-d′ section. The average can be calculated by dividing the sum of the difference between adjacent sampling values with sampling number k, that is, Δ_{α}=[(s[n])−s[n−1])+(s[n−1])−s[n−2])+ . . . (s[n−k])−s[n−k−1])]/k. And the slope tracing wave in the next signal section d′-e′ is increased by the absolute number of slope average |Δ_{α}| at every sampling value, that is, a[n]=a[n−1]+|Δ_{α}|.

This process is repeated while k sampling values are generated and the shape of slope tracing wave is shown as d-e in the drawing 5.

The similar process is repeated until sampling value (point x in the drawing 5) is generated where the amplitude of slope tracing wave becomes equal to or higher than signal amplitude, that is, the slope average □Δ_{α} is recalculated by dividing the sum of the difference between adjacent sampling values in the section d′-e′ with sampling number k at e′ where k-th sampling value is generated.

This recalculated absolute value of slope average |Δ_{α}| refreshes slope tracing wave as shown in e-f by increasing it at every sampling value from slope tracing wave in the section e′-f′, that is, it is refreshed to a[n]=a[n−1]+|Δ_{α}|. When a sampling value is generated at f′ which is the k-th sampling in the section, slope average Δ_{α} is recalculated and slope tracing wave is refreshed at every sampling by adding |Δ_{α}| in the next section.

Just as point x in the drawing 5, if slope tracing wave becomes equal to or higher than signal sampling value while it increases, i.e., s[n]≦a[n−1], the slope tracing wave is refreshed to the amplitude of sampling value, i.e., a[n]=s[n].

Through the procedures described above, the algorithm sets up section p and x which is determined by slope tracing wave as one bend and sets up section x and g as another bend, and applies more sophisticated algorithm which will be described later to each section.

Drawing 6 and drawing 7 shows the operation of ascending slope tracing wave can be varied depending on sampling number k of the p-d section of slope tracing wave. When referring to drawing 3, the slope tracing wave meets a signal at point x by ascending from the lowest value p of the first bend when sampling number k is small. After that, it starts to trace the descending signal at the right side as described above. In this case, two adjacent bends are determined as two separate bend.

However, as shown in the drawing 7, if sampling number k is big, signal amplitude becomes lower than slope tracing wave in the section p-d. In this case, slope tracing wave starts to trace signal amplitude at point x and two bends become one bend, i.e., the first bend is determined to be a part of the second bend. For both descending slope tracing wave and ascending slope tracing wave, the same sampling number k is being used for p-d section and other sections, but it can be different number, if necessary.

Description on the method how to determine signal bend section using above-mentioned descending slope tracing wave and ascending slope tracing wave is followed below:

### Selection of Signal Bend Section Using Descending Slope Tracing Wave

Drawing 8 shows an example determining signal section using descending slope tracing wave. Determine the highest signal point p and point x using slope tracing wave and determine the lowest point v2 by selecting the lowest point of the section or the point where slope change rate is maximum to determine section l2. In addition, section l1 is also determined by finding v1 and the highest point p in the same manner. The highest point p is determined as the highest value at point d where k-th sampling value is generated, and point x is determined as the point where descending slope tracing wave becomes lower than signal amplitude. The l1 and l2 signal can be interpreted by analyzing its shape characteristics. If necessary, l1 and l2 can be defined as one section for signal analysis.

### Selection of Signal Bend Section Using Ascending Slope Tracing Wave

Drawing 9 shows an example determining signal section using ascending slope tracing wave. Determine the lowest signal point p and point x using slope tracing wave and determine the highest point v2 by selecting the highest point of the section or the point where slope change rate is maximum to determine section l1. In addition, section l2 is also determined by finding g and the highest point v in the same manner. The lowest point p is determined as the lowest value at point d where k-th sampling value is generated, and point x is determined as the point where ascending slope tracing wave becomes higher than signal amplitude. The l1 and l2 signal can be interpreted by analyzing its shape characteristics. If necessary, l1 and l2 can be defined as one section for signal analysis.

### Selection of Signal Bend Section Using Both Descending Slope Tracing Wave and Ascending Slope Tracing Wave

Not like Drawing 8 and Drawing 9, section l1 and l2 can be determined by applying both descending slope tracing wave and ascending slope tracing wave. This is shown in Drawing 10.

Drawing 10 shows the method to select signal bend section using both descending slope tracing wave and ascending slope tracing wave. The l1 and l2 can be analyzed independently as before or they can be considered as one section for analysis.

### Selection of Signal Bend Section Using Forward or Reverse Slope Tracing Wave

Drawing 8 shows the method how to determine signal bend section using descending slope tracing wave. Drawing 11 shows the method how to determine signal bend section by generating reverse descending slope tracing wave at the highest point p when descending slope tracing reaches at point d and before the next signal sampling is generated. In Drawing 11, when k-th sampling value is generated after the highest signal point p, the slope tracing wave is refreshed to the point d, and the algorithm calculated average slope Δ_{α}.

After that, as shown in Drawing 11, the algorithm determines the point x′ where slope tracing wave becomes lower than signal sampling value by applying reverse descending slope tracing wave at the highest value point p before the next signal sampling value is generated. And it determines the left end of the bend section by selecting v1 which is the lowest value or where slope change is maximum between the point x′ and the highest point p.

In this case, it is assumed that a number of generated sample values are stored in a memory in the sequence of its generation. When a sampling value is generated after the point d, apply forward descending slope tracing wave to find the point x and set up v2 as the right side of the bend section. This completes the determination step of section l1 of the bend.

The above-mentioned signal section determination method using forward and reverse descending slope tracing wave can be applied to ascending slope tracing wave in the same manner as shown in the Drawing 12.

Hereinafter, detail description on the method how to extract low frequency or high frequency signal using slope tracing wave is followed by referring to Drawing 13 or Drawing 22, i.e., description on the method how to extract frequencies from a signal where more than two frequencies are mixed using above-mentioned slope tracing wave and signal bend section determination method.

Drawing 13 shows a signal where two signals are mixed. A high frequency component is overlapped onto a low frequency sinusoidal signal. The above-mentioned method is applied to the top signal to extract two signal components at the bottom.

Drawing 14a shows the first step of the algorithm which extracts a signal using two descending slope tracing wave. The algorithm uses two descending slope tracing waves which have different number of sampling values for the p-d section. Two descending slope tracing waves are refreshed while tracing a signal from the point a to the point p.

When two slope tracing waves reach at the point p, one maintains long p-d section and the other maintains short p-d section and then they start to descend while tracing a signal and they meet the point x. When the last sampling value of the long maintaining time is generated, the slope tracing wave is refreshed to its prior slope (d point). After that, the algorithm goes through the following steps before the next signal sampling value is generated.

First of all, check if the slope tracing wave which has short maintaining time meets a signal in the section p-d. If it does not meet the signal in the section, the algorithm enables two slope tracing waves to trace the signal continuously. However, as shown in the Drawing 14a, if the slope tracing wave which has short maintaining time meets a signal at the point x in the section p-d, the algorithm determines max. Slope-transition point t between the point x and the point p and sets it up as the right end of the section.

In addition, as shown in the Drawing 14b, reverse descending slope tracing wave is applied at the point p to find the point x′ where the slope tracing wave meets the signal and the algorithm determines max. Slope-transition point t′ between the point x′ and the point p and sets it up as the left end of the section, and then remove the left and right section from the original signal as shown in the Drawing 17a.

Drawing 15a shows the first step of the algorithm which extracts a signal using two ascending slope tracing wave. The algorithm uses two ascending slope tracing waves which have different number of sampling values for the p-d section. Two ascending slope tracing waves are refreshed while tracing a signal from the point a to the point p.

When two slope tracing waves reach at the point p, one maintains long p-d section and the other maintains short p-d section and then they start to ascend while tracing a signal and they meet the point x. When the last sampling value of the long maintaining time is generated, the slope tracing wave is refreshed to its prior slope (d point). After that, the algorithm goes through the following steps before the next signal sampling value is generated.

First of all, check if the slope tracing wave which has short maintaining time meets a signal in section p-d. If it does not meet the signal in the section, the algorithm enables two slope tracing waves to trace the signal continuously. However, as shown in the Drawing 15a, if the slope tracing wave which has short maintaining time meets a signal at the point x in the section p-d, the algorithm determines max. Slope-transition point t between the point x and the point p and sets it up as the right end of the section.

In addition, as shown in the Drawing 15b, reverse ascending slope tracing wave is applied at the point p to find the point x′ where the slope tracing wave meets the signal and the algorithm determines max. Slope-transition point t′ between the point x′ and the point p and sets it up as the left end of the section, and then remove the left and right section from the original signal as shown in the Drawing 17b.

Drawing 16a shows the first step of the algorithm which extracts a signal using two ascending slope tracing waves and two descending slope tracing waves. The algorithm uses total 4 slope tracing waves: two ascending slope tracing waves which have different number of sampling values for the p-d section and two descending slope tracing waves which have different number of sampling values for the p-d section. In the Drawing 16a, two descending slope tracing waves are displayed in a continuous line and two ascending slope tracing waves are displayed in a dotted line. The slope tracing wave which has more sampling values in the section p-d is shown in a thick and light line, and the slope tracing wave which has less sampling values in the section p-d is shown in a thin and dark line. Two different descending slope tracing waves are shown in a dotted line. In addition, the signal is a curve in p-t′, t′-q, q-t, and t-r sections and a curve after the point r which are overlapped with ascending slope tracing wave as shown in the Drawing 16b, and 4 slope tracing waves are refreshed whenever a signal sampling value is generated.

When looking at the operation of descending slope tracing wave first, as shown in the Drawing 16a, when two descending slope tracing waves reach at the point p, one maintains long p-dd1 section and the other maintains short p-d section. When the last sampling value of long maintaining time is generated, the slope tracing wave is refreshed to the prior slope (dd1 point). After that, the algorithm goes through the following steps before the next signal sampling value is generated.

First of all, check if the slope tracing wave which has short maintaining time meets a signal in section p-dd1. As shown in the Drawing 16a, if it does not meet the signal in the section p-dd1, the algorithm enables two slope tracing waves (displayed in a continuous line) to trace the signal continuously. When they meet high frequency bend in t′-q′-t, they start to increase because signal amplitude is increasing. When two descending slope tracing waves reach at the point q, one maintains long p-dd2 section and the other maintains short p-d section, and then they meet the point x while they are descending and tracing the signal as shown in the Drawing. At this point, the algorithm determines max. Slope-transition point t between the point x and the point p and sets it up as the right end of the section.

In addition, as shown in the Drawing 16b, reverse ascending slope tracing wave is applied at the point q to find the point x′ where the slope tracing wave meets the signal and the algorithm determines max. Slope-transition point t′ between the point x′ and the point q and sets it up as the left end of the section, and then remove the left and right section from the original signal as shown in the Drawing 17c.

When looking at the operation of ascending slope tracing wave, because signal amplitude decreases in the p-t′ section of the signal, two ascending slope tracing waves are refreshed to the signal amplitude. When a signal sampling value is generated at the point t′, the two ascending slope tracing wave maintain each different p-d sections. The ascending slope tracing wave which has short maintaining time (displayed in a thin and dark dotted line in the Drawing 16b) maintains short p-d after the point t′ and ascends by tracing the signal. Meanwhile, the ascending slope tracing wave which has long maintaining time (displayed in a thick and light dotted line) moves as described in the Drawing 7 because the signal amplitude becomes lower than the amplitude of slope tracing wave before the section p-d.

When tracing a signal, either or both of ascending slope tracing wave or/and descending slope tracing wave can be used, and each type can has many tracing waves which have each different maintaining time. The number or type of slope tracing waves to use may be determined based on signal characteristics, simplicity requirements of algorithm, and accuracy, etc. but it is generally recommended to use both ascending slope tracing wave and descending slope tracing wave.

Drawing 17a and 17c shows a waveform in which high frequency component is removed. Hereinafter, description on the method how to recover a segmented part of extracted waveform or approximate it to a sinusoidal wave as shown in the Drawing 17a is followed:

As shown in the Drawing 18, low frequency component can be recovered by connecting a segmented part of a signal with a straight line or using various interpolation methods. Drawing 18 shows the low frequency waveform recovered using interpolation for the segmented part of a signal which is shown in the Drawing 17a. Hereinafter, description on the method how to approximate an extracted waveform to a sinusoidal wave is followed:

Drawing 19 shows approximation of an extracted waveform to two different sinusoidal waves. The waveform can be approximated to a part of two different sinusoidal waves at the left side and right side using the max value as a reference. The left one is a sinusoidal wave with amplitude A1 and frequency 1/4T1, and the right one is a sinusoidal wave with amplitude A2 and frequency 1/4T2. However, the accuracy of approximated sinusoidal waves can be varied depending on signal characteristics and there may be considerable errors.

Drawing 20 shows an example of approximation to a sinusoidal wave using extracted three points. Description on the algorithm used to approximate an extracted waveform to a sinusoidal wave which has a minimum error by using three extracted points is followed:

To explain this algorithm, select three points a, b, and c which satisfy phase angle difference □θ_{b}−θₐ=θ_{c}−θ_{b} on a circle whose radius is A as shown in the Drawing 21.

Here, h1, h2, and h3 are the amplitudes at phase angles □θₐ, □θ_{b}, and □θ_{c} when it is assumed that the sinusoidal is sine wave.

Therefore, at the point a, the sine wave is

$$sin ( {θ}_{a} ) = \frac{{h}_{1}}{A} , cos ( {θ}_{a} ) = \sqrt{1 - \frac{{h}_{1}^{2}}{{A}^{2}}} = \sqrt{\frac{{A}^{2} - {h}_{1}^{2}}{{A}^{2}}} \qquad [ eq . 1 ]$$

At the point b, the sine wave is

$$sin ( {θ}_{b} ) = \frac{{h}_{2}}{A} , cos ( {θ}_{a} ) = \sqrt{1 - \frac{{h}_{2}^{2}}{{A}^{2}}} = \sqrt{\frac{{A}^{2} - {h}_{2}^{2}}{{A}^{2}}} \qquad [ eq . 2 ]$$

At the point c, the sine wave is

$$sin ( {θ}_{c} ) = \frac{{h}_{3}}{A} , cos ( {θ}_{a} ) = \sqrt{1 - \frac{{h}_{3}^{2}}{{A}^{2}}} = \sqrt{\frac{{A}^{2} - {h}_{3}^{2}}{{A}^{2}}} \qquad [ eq . 3 ]$$

When applying sin to both sides, θ_{b}−θₐ=θ_{c}−θ_{b} becomes
sin(θ_{b}−θₐ)=sin(θ_{c}−θ_{b}) [eq. 4]

And this becomes
sin(θ_{b})cos(θₐ)−cos(θ_{b})sin(θₐ)=sin(θ_{c})cos(θ_{b})−cos(θ_{c})sn(θ_{b}) [eq. 5]

When applying eq. 1, 2, and 3, this equation becomes

$$\frac{{h}_{2}}{A} \sqrt{\frac{{A}^{2} - {h}_{1}^{2}}{{A}^{2}}} - \frac{{h}_{1}}{A} \sqrt{\frac{{A}^{2} - {h}_{2}^{2}}{{A}^{2}}} = \frac{{h}_{3}}{A} \sqrt{\frac{{A}^{2} - {h}_{2}^{2}}{{A}^{2}}} - \frac{{h}_{2}}{A} \sqrt{\frac{{A}^{2} - {h}_{3}^{2}}{{A}^{2}}} \qquad [ eq . 6 ]$$

This becomes

$${h}_{2} ( \sqrt{{A}^{2} - {h}_{1}^{2}} + \sqrt{{A}^{2} - {h}_{3}^{2}} ) = ( {h}_{1} + {h}_{3} ) \sqrt{{A}^{2} - {h}_{2}^{2}} \qquad [ eq . 7 ]$$

Therefore, the amplitude of the sinusoidal wave is

$$t A = 2 {h}_{2} \sqrt{\frac{{h}_{1} {h}_{3} - {h}_{2}^{2}}{( {h}_{1} + {h}_{3} + 2 {h}_{2} ) ( {h}_{1} + {h}_{3} - 2 {h}_{2} )}} \qquad [ eq . 8 ]$$

The phase values of the point a, b, and c are

$$t {θ}_{a} = {sin}^{- 1} ( \frac{{h}_{1}}{A} ) {θ}_{b} = {sin}^{- 1} ( \frac{{h}_{2}}{A} ) {θ}_{c} = {sin}^{- 1} ( \frac{{h}_{3}}{A} ) \qquad [ eq . 9 ]$$

And the frequency of the sinusoidal wave is

$$f = \frac{( {θ}_{c} - {θ}_{b} ) \cdot {f}_{s}}{2 π ( {n}_{c} - {n}_{b} )} \qquad [ eq . 10 ]$$

Or

$$f = \frac{( {θ}_{b} - {θ}_{a} ) \cdot {f}_{s}}{2 π ( {n}_{b} - {n}_{a} )} \qquad [ eq . 11 ]$$

Meanwhile, if eq.8, 9, 10, and 11 are used, a sinusoidal wave which can be approximated to sampling values can be calculated when Drawing 19 and 3 sampling values in the signal section T1 are given. The amplitude, phase, and frequency can be determined by calculating averages of amplitudes, phases, and frequencies obtained from three sample values of several group calculated at other positions.

Finally, a high frequency signal can be extracted by subtracting the low frequency signal extracted from the original signal by the above-mentioned method. Until now, low frequency signal extraction method using descending slope tracing wave are described, but low frequency or high frequency signal can be extracted even when only ascending slope tracing wave is used or even when both descending and ascending slope tracing waves are used simultaneously.

Drawing 22 and Drawing 23 show an example in which low frequency component is removed from electrocardiogram diagram according to the method in this invention. Electrocardiogram which has sinusoidal baseline fluctuation is introduced is shown at the top, extracted low frequency baseline is shown at the middle, and electrocardiogram after the baseline is removed according to the proposed method in this invention is shown at the bottom.

Hereinafter, detail description on the method how to extract P wave and T wave of electrocardiogram using the above-mentioned slope tracing wave. If it is assumed electrocardiogram signal is s(n) at an arbitrary time, the D_a_s(n) which is the difference between s(n) and its ascending slope tracing wave is as below:
D_{—}a_{—}s(n)=s(n)−a(n) [eq. 12]

Drawing 24a shows original electrocardiogram signal s(n) and a(n). In the difference signal calculated from eq.12, the waveform whose amplitude is over 70% of the highest value is determined as QRS wave, and the distance between the lowest points at both ends is determined as the width of QRS wave, and the left point is called ns and the right point is called ne. Drawing 24c shows D_a_s(n) according to this invention, and QRS wave is removed by the above-mentioned method to emphasize P wave and T wave.

When applying descending slope tracing wave d(n) to the D_a_s(n) waveform in eq. 12, the difference signal between two waveforms is calculated as below.
D_{—}d_{—}s(n)=d(n)−D_{—}a_{—}s(n) [eq. 13]

Drawing 24b shows both D_a_s(n) and descending slope tracing wave d(n) and Drawing 24d shows a drawing where QRS wave is removed from the difference signal by using the above-mentioned method.

Finally, add the two difference signals calculated above, i.e., eq.12 and eq.13, to determine P wave and T wave. Drawing 24c shows the added signal. The P wave is determined as 95% of the max. amplitude within 200 ms before R wave, and the T wave is determined as 95% of the max. amplitude within 400 ms after R wave.

The description until now covered the characteristics and technical advantages of this invention a little bit broadly to help understanding of patent claims which will be presented later. The additional characteristics and advantages which comprise of the patent claims of this invention is followed below. The fact that the proposed concepts and specific examples of this invention can be immediately applied to the design and modification of other structures which are intended to achieve the objectives which are similar to this invention should be recognized by the professionals in this field.

In addition, the disclosed concepts and specific examples of this invention can be used by the professionals in this field as a basis for the design and modification of other structures which are intended to achieve the objectives which are similar to this invention. Furthermore, the equivalent structures modified or changed by the professionals in this field can be varied, replaced, or revised within the limit of the concepts and scope of this invention described in its patent claims.

[ ]

As described above, this invention proposed the method how to extract low frequency signal or high frequency signal from an arbitrary signal by using two slope tracing waves.

One or more than two slope tracing waves are used to determine the high frequency signal section of an arbitrary signal and the signal is removed based on its signal characteristics. And then, low frequency signal is removed by connecting the removed part using interpolation or by approximating it to a sinusoidal wave. In addition, this invention helps easy detection of P wave and T wave from electrocardiogram by using the difference between an arbitrary signal and two slope tracing waves in tracing signal bend. The methods in this invention comprise of distinguishing signal bend, distinguishing and removing baseline fluctuation which has low frequency components by using the shape characteristics of the distinguished section. This method also helps easy detection P wave and T wave from electrocardiogram no matter what baseline fluctuation is introduced or not.

What is claimed:

1. A method for extracting a high frequency and low frequency component from a signal, comprising:
(a) Checking if a 1^{st} descending slope tracing wave d1(n) meets signal s(n) in a section of k2 in which a 2^{nd} descending slope tracing wave d2(n) maintains a function value when the signal s(n) reaches a maximum value (p point) at an nth sample point;
(b) identifying a max. Slope-inversion point t in a section of [n, n+k2] when there is a cross point (point x) where d1(n) and s(n) meet each other in operation (a), and setting the max. Slope-inversion point t as a right most point of the section;
(c) reverse descending slope tracing wave when there is a cross point (point x) where d1(n) and s(n) meet each other in operation (a), the reverse descending is applied at the nth sample point (p point) to find x′ point where the reverse descending slope tracing wave meets with signal s(n) and determines a max. Slope-inversion point t′ of a signal between x′ point and p point, and setting the max. Slope-inversion point t′ as a left most point of the section:
(d) extracting left and right section waveforms determined as a right most end and a left most end from the original signal:
(e) connecting the right most end and the left most end of the section.

2. A method for extracting a high frequency and low frequency component from a signal, comprising:
(a) Checking if also ascending slope tracing wave a1(n) meets signal s(n) in a section of k2 in which a 2^{nd} ascending slope tracing wave a2(n) maintains a function value when the signal s(n) reaches a maximum value (p point) at an nth sample point:
(b) When there is a cross point (point x) identifying a max. Slope-inversion point t in a section of [n, n+k2] where a1(n) and s(n) meet each other in operation (a), and setting the max. Slope-inversion point t as a right most point of the section:
(c) reverse descending slope tracing wave when there is a cross point (point x) where a1(n) and s(n) meet each other in operation (a), the reverse descending is applied at the nth sample point (p point) to find x′ point where the reverse descending slope tracing wave meets with signal s(n) and determines a max. Slope-inversion point t′ of a signal between x′ point and p point, and setting the max. Slope-inversion point t′ as a left most point of the section:
(d) extracting left and right section waveforms determined as a right most end and a left most end from the original signal:
(e) connecting the right most end and the left most end of the section.

3. A method connecting the right end and the left end of the section by a straight line or interpolation for either claim 1 or claim 2.

4. A method connecting the right end and the left end of the section by using approximation to a sinusoidal wave for either claim 1 or claim 2.

5. A method of claim 1 or claim 2, further comprising:
selecting a signal bend using one of ascending slope tracing wave descending slope tracing wave or a combination thereof prior to operation (a).

6. A method connecting the right end and the left end of the section using approximation to a sinusoidal wave for either claim 1 or claim 2, and also determining amplitude, phase, and frequency from three sample values.

7. A method using two descending slope tracing waves which have two different value k1 and k2 (k1<k2), to extract a low frequency component and a high frequency component from a signal s(n) which is sampled at a fixed interval for an original signal which has more than two frequency components, the method including using two ascending slope tracing waves which have two different value k1 and k2 (k1<k2), to extract the low frequency component and the high frequency component from the s(n) which is sampled at a fixed interval for the original signal which has more than two frequency components.

8. The method of any of claims 1, 2, and 7, wherein the original signal is an electrocardiogram signal and the method extracts and detects P wave and T wave from the electrocardiogram signal.
